(19) United States
(12) Patent Application Publication
(10) Pub. No.: US 2019/0223848 A1
(43) Pub. Date: Jul. 25, 2019

(54) NON-INVASIVE OCULAR BIOLOGICAL MATERIAL GATHERING DEVICE

(71) Applicants: Harvey Fishman, Menlo Park, CA (US); Vera B. Morhenn, Woodside, CA (US); Jonathan N. Mansbridge, Woodside, CA (US)

(72) Inventors: Harvey Fishman, Menlo Park, CA (US); Vera B. Morhenn, Woodside, CA (US); Jonathan N. Mansbridge, Woodside, CA (US)

(21) Appl. No.: 15/877,456

(22) Filed: Jan. 23, 2018

(51) Int. Cl. A61B 10/02; A61B 3/00; A61B 3/14

(52) U.S. Cl. CPC A61B 10/02; A61B 10/0045; A61B 3/145; A61B 3/0008

(57) ABSTRACT

The biological material gathering device disclosed herein is designed for non-invasive isolation of biological material from an eye, eyelid, or periocular. The device uses a plurality of adhesive beads engaged on a microfiber bundle to harvest biological materials from an individual without the need for penetrating or invasive methods. Such biological materials may then be processed and examined to determine the etiology of pathologies or malignancies affecting the individual. The biological material gathering device preferably comprises a tonometer tip base and housing for use in commonly-available ophthalmological examination devices. The present invention may further comprise video imaging capability so as to register the precise and relative locations of harvested biological materials for later reference. Finally, the biological material gathering device disclosed herein may be made available in kit form for clinical use.

### PRIORITY NOTICE

The present application makes no claims of priority under 35 U.S.C. § 119(e) to any U.S. Provisional Patent Applications.

### CROSS REFERENCE TO RELATED PATENT APPLICATIONS

The present application makes no reference to any other related filed patent applications.

### STATEMENT REGARDING FEDERAL SPONSORSHIP

No part of this invention was a result of any federally sponsored research.

### TECHNICAL FIELD OF THE INVENTION

The present invention relates in general to biological material gathering devices and, more specifically, to a biological material gathering device for non-invasive isolation of biological material from an eye, eyelid, or periocular area.

### COPYRIGHT AND TRADEMARK NOTICE

A portion of the disclosure of this patent application may contain material that is subject to copyright protection. The owner has no objection to the facsimile reproduction by anyone of the patent document or the patent disclosure, as it appears in the Patent and Trademark Office patent file or records, but otherwise reserves all copyrights whatsoever.

Certain marks referenced herein may be common law or registered trademarks of third parties affiliated or unaffiliated with the applicant or the assignee. Use of these marks is by way of example and should not be construed as descriptive or to limit the scope of this invention to material associated only with such marks.

### BACKGROUND OF THE INVENTION

Biological material may be any material produced by or present in a living organism. In terms of the present disclosure, biological material may especially refer to a polynucleotide, polypeptide, polysaccharide, lipid, protein, small organic or inorganic molecule, toxin, neurotransmitter, amino acid, neuropeptide, bacteria, virus, parasite, or other similar molecule. Such biological materials are known to be present on an eye, tear film, eyelid, or periocular area of a human organism, and variations in concentrations of such materials may be correlated with various benign and pathological conditions present in such an organism.

The conjunctiva of an eye, that mucous membrane lining the anterior surface of the eye and the interior surface of the eyelids, comprises a highly vascularized epithelium that is easily accessible for imaging studies and is a common source of clinical eye-related complaints. The anterior eye surface is especially exposed to exogenous agents, and may reliable exhibit early symptoms of various infections, irritations, and reactions affecting the surrounding anatomy. The conjunctiva may also reliably indicate the presence of endogenous sources of pathology, for example changes in vascularization related to diabetes or hypertension, and may further express biological materials correlated with various malignancies or subclinical infections. Such pathologies may be sampled or biopsied from the eye using various methods.

It is known to have an eye sampling method using a plurality of medical instruments to create an incision within the surface of the eye to access and vacuum out a sample of aqueous or vitreous humor. Such a method may be known as a “paracentesis” and may be used in sampling ocular biological material for the presence of various malignancies or pathologies. Such a method carries with it those risks inherent in any surgical procedure, such as infection or unintended physical trauma, and may also lead to such complications as retinal detachment, hemorrhage into the eye, or cataract formation. The convalescent phase following such a procedure may involve several weeks of eye drop application and limited weight-bearing by the affected individual. Moreover, there is a high patient barrier to having the eye cut into.

It is known to have an eye sampling method using a thin needle to penetrate the surface of the eye or any pathological cellular mass in the surrounding anatomy and withdraw biological material. Such a method may be known as “fine-needle aspiration” and may be used in sampling any of a plurality of lumps or masses affecting the periocular anatomy of an individual. Such a method is considered a less traumatic alternative to open biopsy, though still carries with it the risk of various complications such as bruising and soreness at the injection site. In the case of tumor sampling, such an invasive procedure may carry the risk of rupturing the tumor sac and disseminating tumor material throughout the body of the affected individual.

It is known to have an eye sampling method using a plurality of medical instruments to section and excise biological material, especially in its entirety. Such a method may be known as “excisional biopsy” and may be used to simultaneously sample a tissue mass (for example a portion of the cornea, conjunctiva, sclera, epi-sclera, choroid, retina, choriocapillaris, optic nerve, or muscle) and effect a cure of the pathology. Such a method, though, is more complicated and extensive than less invasive sampling methods and carries with it those risks and complications inherent in any surgical procedure.

There is a need in the art for a biological material gathering device and method that allows for non-invasive isolation of biological material from an eye, eyelid, or periocular area. Such a device may comprise a bundle of microfibers capped with adhesive beads that may bind various biological materials. Such a device may further comprise a standard tonometer tip base, and may incorporate video imaging capability. Finally, such a device may be made available in kit form for clinical use.

It is to these ends that the present invention has been developed.

### BRIEF SUMMARY OF THE INVENTION

To minimize the limitations in the prior art, and to minimize other limitations that will be apparent upon reading and understanding the present specification, the present invention describes a biological material gathering device.

It is an objective of the present invention to provide a biological material gathering device for non-invasive isolation of biological material from an eye, eyelid, or periocular area.

It is another objective of the present invention to provide a biological material gathering device comprising a plurality of microfibers capped with adhesive beads.

It is another objective of the present invention to provide a biological material gathering device further comprising a standard tonometer tip base.

It is another objective of the present invention to provide a biological material gathering device further comprising video imaging capability.

It is another objective of the present invention to provide a biological material gathering device available in a kit form for clinical use.

These and other advantages and features of the present invention are described herein with specificity so as to make the present invention understandable to one of ordinary skill in the art, both with respect to how to practice the present invention and how to make the present invention.

### BRIEF DESCRIPTION OF THE SEVERAL VIEWS OF THE DRAWINGS

Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention.

FIG. 1 illustrates a single microfiber and bead combination.

FIG. 2 illustrates a single microfiber and adhesive bead combination.

FIG. 3 illustrates a side view of a microfiber and adhesive bead combination bundle.

FIG. 4 illustrates an end view of a microfiber and adhesive bead combination bundle.

FIG. 5 illustrates an Embodiment 001 of a biological material gathering device.

FIG. 6 illustrates an Embodiment 002 of a biological material gathering device.

FIG. 7 illustrates an Embodiment 003 of a biological material gathering device.

FIG. 8 illustrates a relative proportion of isolates sampled from various individuals.

FIG. 9 illustrates a relative comparison of genes expressed by various individuals.

### DETAILED DESCRIPTION OF THE INVENTION

Certain terminology is used in the following description for reference only and is not limiting. The words “front,” “rear,” “anterior,” “posterior,” “lateral,” “medial,” “upper,” “lower,” “outer,” “inner,” and “interior” refer to directions toward and away from, respectively, the geometric center of the invention, and designated parts thereof, in accordance with the present disclosure. Unless specifically set forth herein, the terms “a,” “an,” and “the” are not limited to one element, but instead should be read as meaning “at least one.” The terminology includes the words noted above, derivatives thereof, and words of similar import.

The present invention relates in general to biological material gathering devices and, more specifically, to a biological material gathering device for non-invasive isolation of biological material from an eye, eyelid, or periocular area.

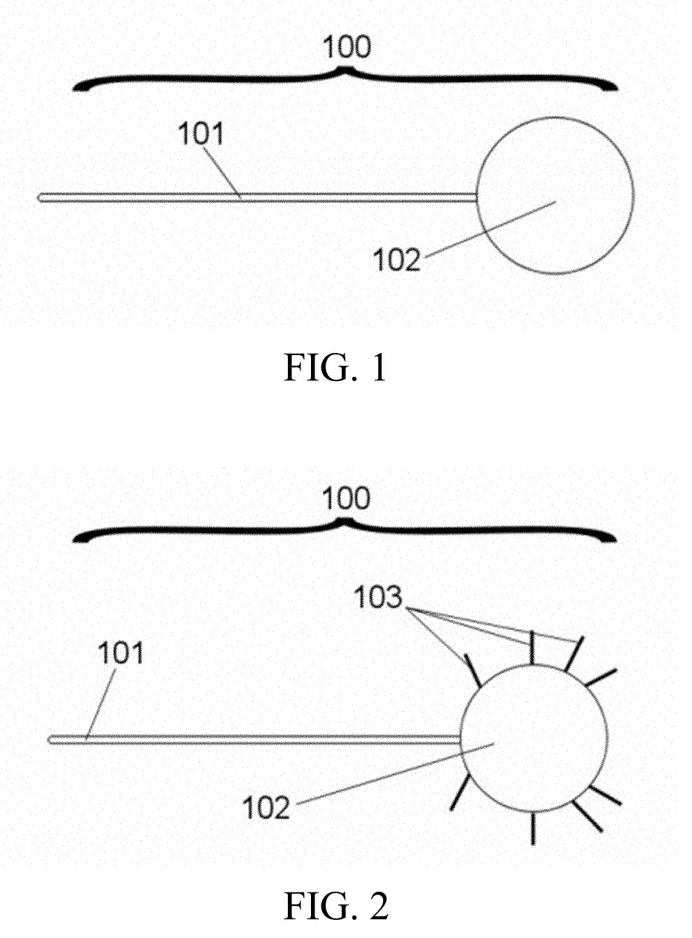

FIGS. 1 and 2 illustrate a microfiber and adhesive bead combination 100 identifying a microfiber 101, an adhesive bead 102, and a plurality of adhesive fibers 103. In a preferred embodiment the microfiber and adhesive bead combination 100 may be touched against an eye, eyelid, or periocular area of an individual and may harvest biological materials from such areas. The microfiber 101 may comprise any appropriate flexible, medical-grade fiber suitable for such use, and may specifically comprise an optical fiber, an electrical fiber, or other mechanical sensing wire. Each microfiber 101 may be designed such they may transmit a signal, whether physically or wirelessly, to an appropriate detector of such a signal.

The adhesive bead 102 may be covered in a plurality of adhesive fibers 103 for binding to various biological materials, and may comprise a plurality of weights or physical properties that may facilitate such binding to a preferred biological material. The adhesive bead 102 may be removably attached to the microfiber 101 to facilitate its release and collection from the system for analysis. Such a bead may be known as a “Lab in a Bead.”

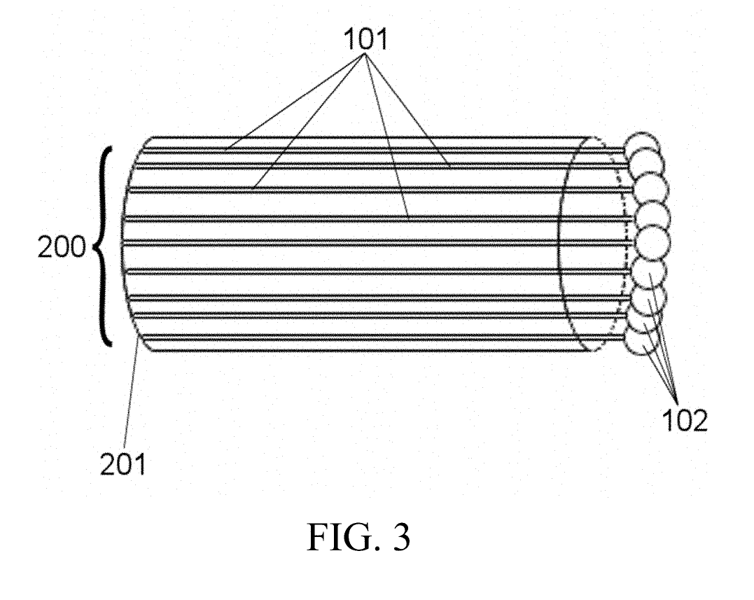

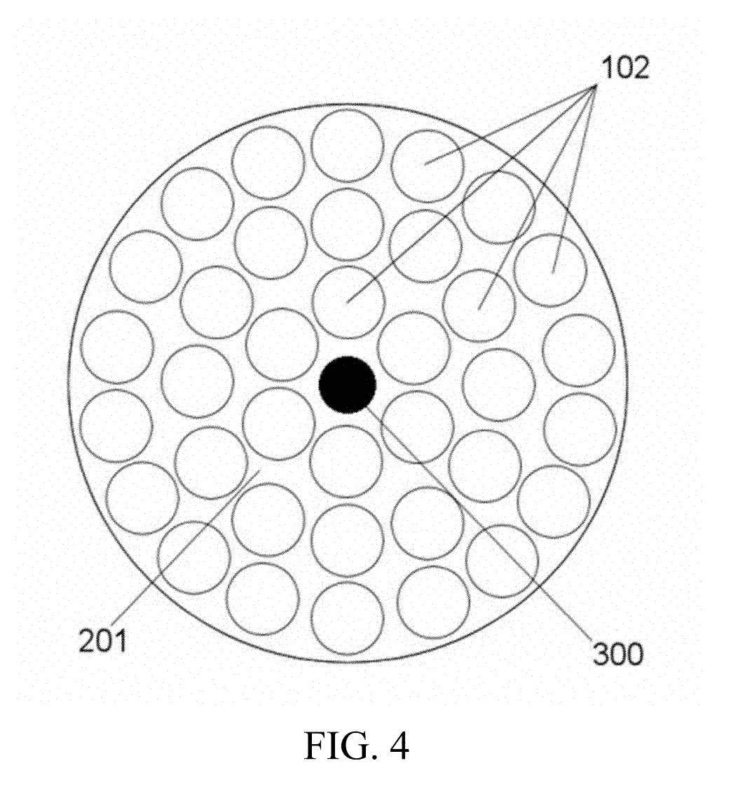

FIGS. 3 and 4 illustrate a microfiber and adhesive bead combination bundle 200 identifying a plurality of microfibers 101, a plurality of adhesive beads 102, a tonometer tip base 201, and an optical fiber 300. In a preferred embodiment the microfiber and adhesive bead combination bundle 200 may facilitate the harvesting of biological materials from an individual by applying a plurality of adhesive beads 102 to the harvesting site. The plurality of microfibers 101 may comprise any appropriate flexible, medical-grade fiber suitable for such use including, but not limited to, an optical fiber, an electrical fiber, other mechanical sensing wire, or any combination thereof. The plurality of adhesive beads 102 may comprise any appropriate weights or physical properties for the harvesting of a preferred biological material, or may comprise a combination of weights or physical properties for the harvesting of a plurality of preferred biological materials. Video imaging may be incorporated into the system through the use of fiber optic technology, with an optical fiber 300 providing remote visualization of the harvesting site. Such video imaging may register the precise and relative locations of biological material harvesting for later reference.

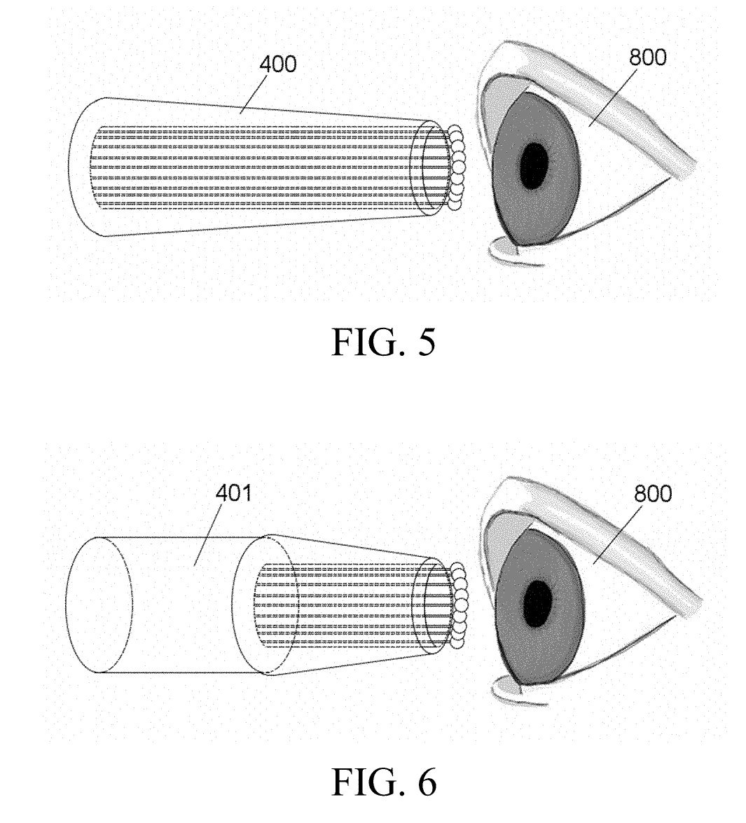

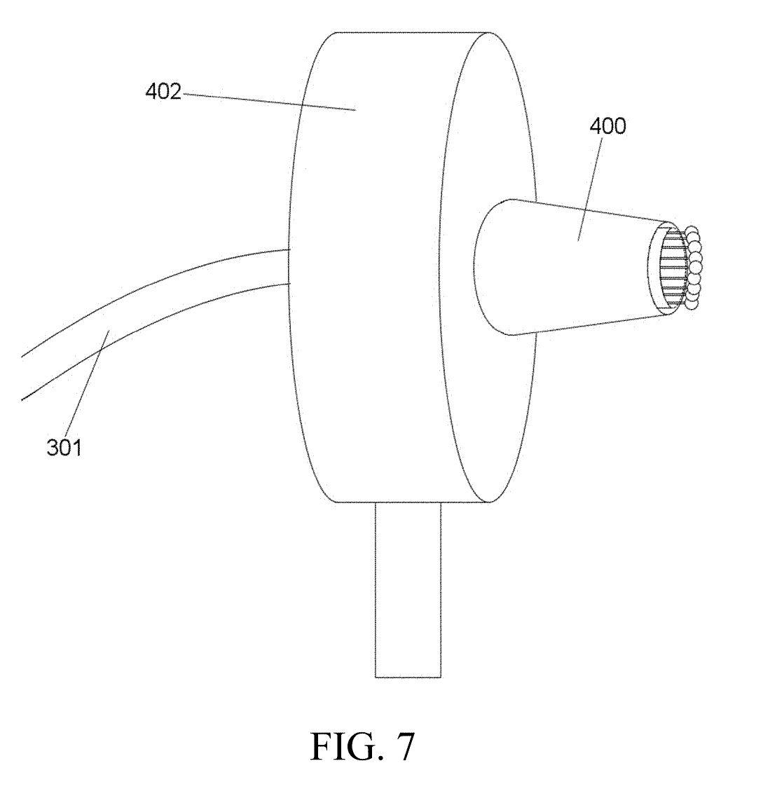

FIGS. 5 and 6 illustrate preferred embodiments of a biological material gathering device identifying a first combination housing 400, a second combination housing 401, and a human eye 800. The various combination housings may be designed as appropriate for their relevant application, though generally comprise a plurality of standardized tonometer tip housings compatible with commonly-available ophthalmological examination devices.

FIG. 7 illustrates a preferred embodiment of a biological material gathering device identifying a fiber optic bundle 301, a first combination housing 400, and a slit lamp holder 402.

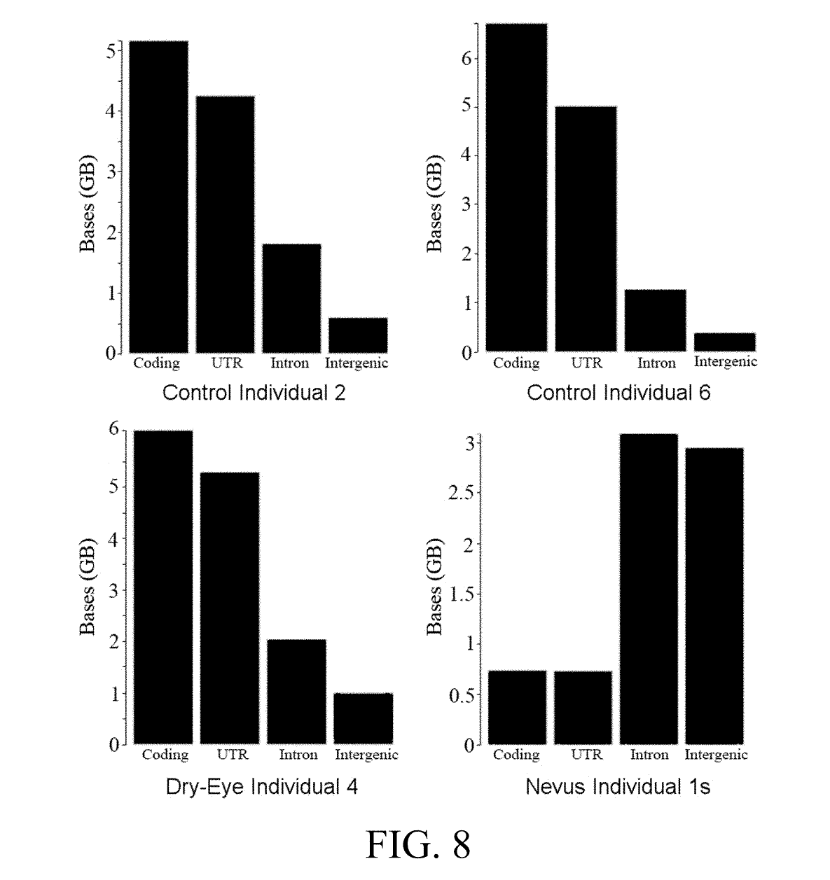

FIG. 8 illustrates a relative proportion of isolates sampled from various individuals. The figure depicts the relative proportion of coding, untranslated region (UTR), intronic and intergenic sequences found in the RNA isolated from samples harvested from two control, one dry eye, and one conjunctival nevus individual. It may be noted that there is a far higher proportion of intronic and intergenic sequences in the sample from the nevus individual. This is partly explained by a large increase in the amount of micro RNA sequences in this sample.

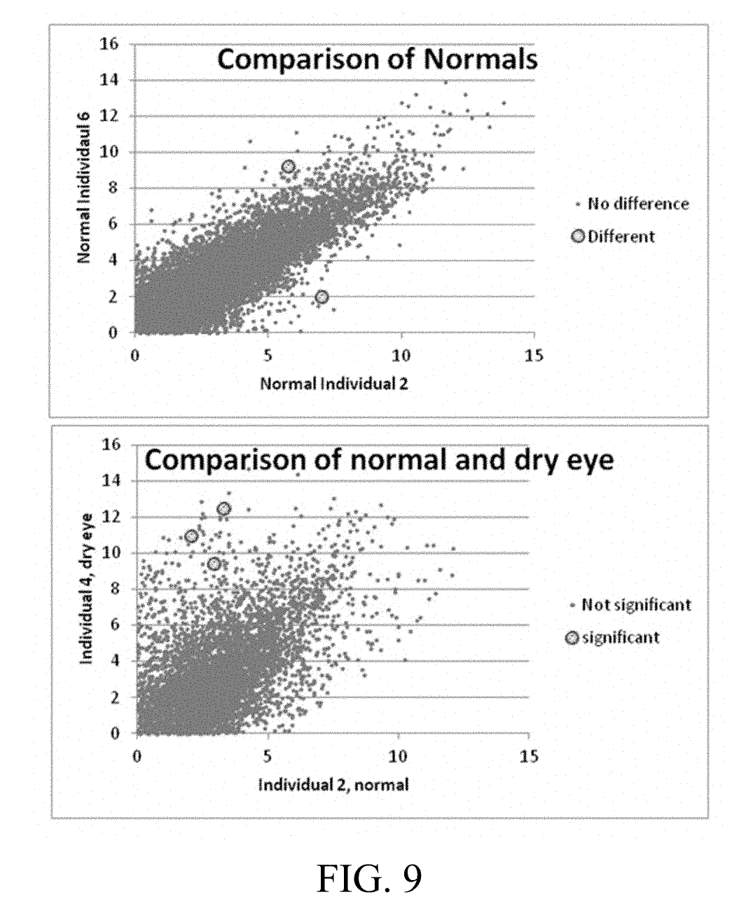

FIG. 9 illustrates a relative comparison of genes expressed by various individuals. The figure depicts comparisons in gene expression of samples from a pair of normal individuals (on the left) and between a dry eye patient and one of the normal individuals (on the right). It may be noted that, although very few of the comparisons were significantly different, there is clearly a much broader variation in gene expression in the samples from the dry eye patient.

The invention provides a non-invasive method for collecting biological materials, such as polynucleotide, from the cells or fluid on the eye. These biological materials can then be characterized to indicate the presence of a local or systemic response in the subject. In one embodiment, samples containing nucleic acids are obtained non-invasively. In another embodiment, the invention provides a method for diagnosing various diseases of the eye, such as melanoma, squamous cell carcinoma, and basal cell carcinoma. The invention also provides a method for diagnosing various inflammatory eye diseases, such as conjunctivitis, as well as for diagnosing disorders of the eye, such as dry eye.

As used herein, the term “biological material” includes a plurality of materials that have biological activity or play a biological role. For example, biological material includes polynucleotides such as DNA, RNA, mRNA, and cDNA, polypeptides such as the various interleukins, lipids such as cholesterol, fatty acids, inflammatory mediators such as leukotrienes, prostaglandins, and others. Bacterial and viral products, such as lipopolysaccharides, may also be included in this group.

As used herein, the terms “nucleic acid,” “polynucleotide,” or “nucleic acid sequence” refer to a polymer of deoxyribonucleotides or ribonucleotides, in the form of a separate fraction or as a component of a larger construct. Polynucleotide or nucleic acid sequences of the invention include DNA, RNA, mRNA, miRNA, other RNAs, coding and non-coding DNA, and cDNA sequences.

As used herein, the term “polypeptide” refers to a polymer of amino acid residues in the form of a separate fragment or component of a larger construct. An example of a polypeptide includes the amino acid sequences encoding a cytokine or fragments thereof. A polypeptide may encode for a functional protein or fragments of a protein. For example, an IL-4 polypeptide includes the full length protein sequence of IL-4 and fragments thereof consisting of a polymer of amino acids.

“Cytokine” as used herein means any number of factors that play a role in cellular regulation or differentiation. For example, cytokines can include the family of interleukins (IL) including IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23 and so forth, as well as, factors belonging to the transforming growth factor beta (TGF-beta) superfamily, GM-CSF, chemokines, other secreted and cell surface signaling molecules, and interferons.

The term “sample” refers to any preparation derived from the eye of a subject. For example, a sample of cells, exosomes, or tear fluid obtained using the non-invasive method described above, may be used to isolate polynucleotides, polypeptides, DNA, or lipids. In addition, the method of the invention can be used in vitro, for example with cells from the eye cultured on a solid or semi-solid support and organotypic eye constructs. In such instances, the eye cells may be from any source. Biological material obtained from any in vitro or in vivo specimen, in purified or non-purified form, can be used as the starting material for detection of a biological activity, such as an infection, provided it contains the biological material of interest. For example, a sample may be used to detect an infection by detecting polynucleotides, provided it contains, or is suspected of containing, the specific polynucleotide sequence encoding a polypeptide, such as a cytokine, which is indicative of an eye infection.

Samples from a tissue may be isolated by any number of means well known in the art. Invasive methods for isolating a sample include the use of needles, for example during blood sampling, as well as biopsies of various tissues. Due to the invasive nature of these techniques there is an increased risk of morbidity and mortality. The present invention provides a method and kit useful for non-invasively obtaining a sample which may be used as a source for obtaining biological factors in the detection, diagnosis, or prognosis of various diseases, disorders, or inflammatory reactions. In a preferred embodiment, the invention consists of a non-invasive method for obtaining an eye sample for use in isolating biological factors, for example nucleic acids or polypeptides, to detect an inflammatory reaction. In this embodiment, cells from the eye may be removed by using an adhesive tape, for example Duct tape (333 Duct tape Nashua tape products) or Scotch® tape (3M Scotch 810, St. Paul, Minn.). However, a preferred method is to use D-SQUAME® (CuDerm, Dallas Tex.) to strip the conjunctiva of the eye. In this embodiment, the eye is stripped with the tape and the stripped cell(s) and cellular material are then recovered from the tape or other items. In another embodiment, a tonometer may be tipped with a porous device that can be treated in the same way. For example, tape used to obtain eye cells and cellular material may be centrifuged in a sterile microfuge tube containing lysis buffer. In the case of the tonometer, the cells and cellular material on the tip of the tonometer may be transferred to any appropriate sterile container, such as a centrifuge tube, Petri dish containing the lysis buffer and any cells present lysed therein with lysis buffer, or a shipping container. The same lysis buffer may be used for each piece of tape or tonometer tip used at a single site of the conjunctiva. The sample obtained may then be further processed, for example, to isolate nucleic acids, polypeptides, lipids, or other molecules. Preferably, the method utilized does not affect the amount of nucleic acids, polypeptides, lipids, or other molecules being measured. The invention provides a rapid, non-invasive method for obtaining polynucleotides, such as mRNA, which are helpful to establish changes in the synthetic patterns of the eye's cells. Using the stripping methods of the present invention the presence of a local or systemic disease, disorder, or inflammatory reaction may be identified, distinguished, or diagnosed, including genetic diseases. In the invention, any reaction, disease, or disorder that corresponds to an induction of transcription and polypeptide synthesis may be detected by the methods of the invention.

Polynucleotides can be isolated from the lysed cells and cellular material by any number of means well known to those skilled in the art. For example, a number of commercial products are available for isolating polynucleotides, including but not limited to, TriReagent (Molecular Research Center, Inc., Cincinnati, Ohio) may be used. The isolated polynucleotides can then be tested or assayed for particular nucleic acid sequences, including a polynucleotide encoding a cytokine or other protein.

In another embodiment, polypeptides may be obtained from the sample by methods known to those skilled in the art. For example, gross preparations of cells obtained using the non-invasive techniques of the invention contain polypeptides. The polypeptides may be further isolated, purified, detected, or measured using conventional means including preparative chromatography and immunological separations involving monoclonal or polyclonal antibodies or other proteomic methods known to those skilled in the art. The polypeptides may then be characterized to indicate the presence of an eye-related reaction.

In one embodiment, the invention provides a method for distinguishing an irritant reaction from an allergic reaction in a sample from the eye by detecting a polynucleotide encoding a cytokine or other protein. The relative quantity of certain cytokines with respect to a normal or standard tissue sample distinguishes the type or reaction and/or the reaction's severity. While existing clinical tests may not be able to distinguish an irritant reaction from an allergic reaction in the tissue, the non-invasive method of the present invention is capable of distinguishing between the two reactions by their relative cytokine expression profiles. Irritant reactions in the eye can be distinguished from an allergic conjunctivitis by the presence or absence of a polynucleotide encoding a cytokine or the cytokine polypeptide. For example, in the present invention, cells obtained from an irritant reaction had undetectable levels of polynucleotide encoding IL-4, compared with polynucleotides from cells of an allergic reaction according to the method used. Consequently, the process may employ, for example, DNA or RNA, including mRNA, microRNA, or other RNA isolated from a tissue. The DNA or RNA may be single stranded or double stranded. When RNA is obtained, enzymes and conditions optimal for reverse transcribing the template to DNA well known in the art can be used. Alternatively, the RNA can be subjected to RNA sequencing (RNASeq), polymerase chain reaction (PCR), a polynucleotide hybridization-based assay, RNAse protection assay, or other suitable methods to characterize and quantify RNA molecules in the sample.

RNA in the sample may be sequenced using a proprietary method (IIlumina SMART-Seqv4 Ultra low Input RNA kit) and the resulting RNA sequence data analyzed by means of the Tuxedo suite of sequence analysis programs such as Tophat and Cufflinks. Expression data may be filtered to identify marker sequences, RNA processing, such as splicing, patterns and genetic changes. Changes in the expression of genes related to specific cellular processes, such as innate and adaptive immune responses, may be identified and characterized.

A DNA-RNA hybrid that contains one strand of each polynucletide may also be used. A mixture of polynucleotides may also be employed, or the polynucleotides produced in a previous amplification reaction, using the same or different primers may be used. In the instance where the polynucleotide sequence is to be amplified, the polynucleotide sequence may be a fraction of a larger molecule or can be present initially as a discreet molecule, such as the specific sequence in the entire nucleic acid. It is not necessary that the sequence to be amplified is present initially in a pure form; it may be a minor fraction of a complex mixture, such as contained in whole human DNA.

In another embodiment, the polynucleotide in the sample may be analyzed by Northern or Southern blot. In this technique, the polynucleotides are separated on a gel and then probed with a complementary polynucleotide to the sequence of interest. For example, RNA is separated on a gel transferred to nitrocellulose and probed with complementary DNA to the sequence of interest. The complementary probe may be labeled radioactively, chemically etc. The probe used will be indicative of the presence of the polynucleotide of interest.

Detection of a polynucleotide encoding a cytokine may be performed by standard methods such as size fractionating the nucleic acid. Methods of size fractioning the DNA and RNA are well known to those of skill in the art, such as by gel electrophoresis, including polyacrylamide gel electrophoresis (PAGE). For example, the gel may be a denaturing 7 M or 8 M urea-polyacrylamide-formamide gel. Size fractionating the nucleic acid may also be accomplished by chromatographic methods known to those of skill in the art.

The detection of polynucleotides may optionally be performed by using radioactively labeled probes. Any radioactive label may be employed which provides an adequate signal. Other labels include ligands, color dyes, and fluorescent molecules, which can serve as a specific binding pair member for a labeled ligand, and the like. The labeled preparations are used to probe for a polynucleotide by the Southern or Northern hybridization techniques, for example. Nucleotides obtained from samples are transferred to filters that bind nucleotides. After exposure to the labeled polynucleotide probe, which will hybridize to nucleotide fragments containing target nucleic acid sequences, the binding of the radioactive probes to target nucleic acid fragments is identified by autoradiography (see Genetic Engineering, 1 ed. Robert Williamson, Academic Press (1981), pp. 72-81). The particular hybridization technique used is not essential to the invention. Hybridization techniques are well known or easily ascertained by one of ordinary skill in the art. As improvements are made in hybridization techniques, they can readily be applied to the method of the invention. For instance, microfluidic techniques may be applied to the application of these methods.

The polynucleotides encoding the desired polypeptide may be amplified before detecting. The term “amplified” refers to the process of making multiple copies of the nucleic acid from a single polynucleotide molecule. The amplification of polynucleotides can be carried out in vitro by biochemical processes known to those of skill in the art. The amplification agent may be any compound or system that will function to accomplish the synthesis of primer extension products, including enzymes. Suitable enzymes for this purpose include, for example, E. coli DNA polymerase I, Taq polymerase, Klenow fragment of E. coli DNA polymerase I, T4 DNA polymerase, other available DNA polymerases, mutant or modified polymerase, reverse transcriptase, ligase, and other enzymes, including heat-stable enzymes (i.e., those enzymes that perform primer extension after being subjected to temperatures sufficiently elevated to cause denaturation). Suitable enzymes will facilitate combination of the nucleotides in the proper manner to form the primer extension products that are complementary to each mutant nucleotide strand. Generally, the synthesis will be initiated at the 3′-end of each primer and proceed in the 5′-direction along the template strand, until synthesis terminates, producing molecules of different lengths. There may be amplification agents, however, that initiate synthesis at the 5′-end in the other direction, using the same process as described above. In any event, the method of the invention is not to be limited to the embodiments of the amplification described herein.

One method of in vitro amplification which can be used according to this invention is the polymerase chain reaction (PCR) described in U.S. Pat. Nos. 4,683,202 and 4,683,195. The term “polymerase chain reaction” refers to a method for amplifying DNA base sequence using a heat-stable polymerase and two oligonucleotide primers, one complementary to the (+)-strand at one end of the sequence to be amplified and the other complementary to the (−)-strand at the other end. Because the newly synthesized DNA strands can subsequently serve as additional templates for the same primer sequences, successive rounds of primer annealing, strand elongation, and dissociation produce rapid and highly specific amplification of the desired sequence. The polymerase chain reaction is used to detect the presence of polynucleotides encoding cytokines in the sample. Many polymerase chain methods are known to those of skill in the art and may be used in the method of the invention. For example, DNA can be subjected to 30 to 35 cycles of amplification in a thermocycler as follows: 95° C. for 30 sec, 52° to 60° C. for 1 min, and 72° C. for 5 min. For another example, DNA can be subjected to 35 polymerase chain reaction cycles in a thermocycler at a denaturing temperature of 95° C. for 30 sec., followed by varying annealing temperatures ranging from 54-58° C. for I min, an extension step at 70° for 1 min with a final extension step of 10 minutes at 70° ??C.

Primers for use in amplifying the polynucleotides of the invention may be prepared using any suitable method, such as the conventional method, phosphotriester and phosphodiester methods or automated embodiments thereof so long as the primers are capable of hybridizing the polynucleotides of interest. One method of synthesizing oligonucleotides on a modified solid support is described in U.S. Pat. No. 4,458,066. The exact length of primer will depend on many factors, including temperature, buffer, and nucleotide composition. The primer must prime the synthesis of extension products in the presence of the inducing agent for amplification.

Primers used according to the method of the invention are complementary to each strand of the nucleotide sequence to be amplified. The term “complementary” means that the primers must hybridize with their respective strands under conditions that allow the agent for polymerization to function. In other words, the primers that are complementary to the flanking sequences hybridize with the flanking sequences and permit the amplification of the nucleotide sequence. Preferably, the 3′ terminus of the primer that is extended has perfectly base paired complementarity with the complementary flanking strand.

In addition, RNAse protection assays may be used if RNA is the nucleotide obtained from the sample. In this procedure, a labeled antisense RNA probe is hybridized to the complimentary polynucleotide in the sample. The remaining unhybridized single-stranded probe is degraded by ribonuclease treatment. The hybridized double stranded probe is protected from RNAse digestion. After an appropriate time, the products of the digestion reaction are collected and analyzed on a gel (see for example Ausubel et al, CURRENT PROTOCOLS IN MOLECULAR BIOLOGY, section 4.7.1 (1987)). As used herein, “RNA probe” refers to a ribonucleotide capable of hybridizing to RNA in a sample of interest. Those skilled in the art will be able to identify and modify the RNAse protection assay specific to the polynucleotide to be measured, for example, the probe's specificity may be altered, as well as, the hybridization temperature, the quantity of nucleic acid, etc. Additionally, a number of commercial kits are available, for example, RiboQuant™ Multi-Probe RNAse Protection Assay System (Pharmingen, Inc. San Diego, Calif.).

Those of ordinary skill in the art will know of various amplification methodologies which can also be utilized to increase the copy number of target nucleic acid. The polynucleotides detected in the method of the invention can be further evaluated, detected, cloned, sequenced, and the like, either in solution or after binding to a solid support, by any method usually applied to the detection of a specific nucleic acid sequence such as another polymerase chain reaction, oligomer restriction (Saiki et al., Bio/Technology 3:1008-1012 (1985)), allele-specific oligonucleotide (ASO) probe analysis (Conner, et al., Proc. Natl. Acad. Sci. USA 80: 278 (1983), oligonucleotide ligation assays (OLAs) (Landegren et al., Science 241:1077 (1988)), sequencing, the RNAse Protection Assay and the like. Molecular techniques for DNA analysis have been reviewed (Landegren et al, Science 242:229-237 (1988)). Following DNA amplification, the reaction product may be detected by Southern blot analysis, without using radioactive probes. In such a process, for example, a small sample of DNA containing the polynucleotides obtained from the tissue or subject are amplified, and analyzed via a Southern blotting technique. The use of non-radioactive probes or labels is facilitated by the high level of the amplified signal. In one embodiment of the invention, one nucleoside triphosphate is radioactively labeled, thereby allowing direct visualization of the amplification product by autoradiography. In another embodiment, amplification primers are fluorescently labeled and run through an electrophoresis system. Visualization of amplified products is by laser detection followed by computer assisted graphic display, without a radioactive signal.

Simple visualization of a gel containing the separated products may be utilized to determine the presence or severity of an eye condition/disease. For example, staining of a gel to visualize separated polynucleotides, a number of stains are well known to those skilled in the art. However, other methods know to those skilled in the art may also be used, for example, scanning densitometry, computer aided scanning, and quantitation as well as otherwise.

Thus, the methods described above can be used to non-invasively obtain a sample of tissue from a subject suspected of having an eye disease, such as a nevus or a melanoma, and to isolate polynucleotides from the sample. The polynucleotides can then be analyzed using methods such as, but not limited to, those described above. Any number of cytokine levels can be quantified by measuring their relative expression in the sample obtained and comparing these levels to normal-standard samples, for example, the mRNA level(s) in a cell change when the production of proteins in the eye is either increased or reduced. Thus, a measurement of RNA, in particular mRNA, provides a monitor of events such as inflammatory processes occurring in the eye or as a result of a local or systemic response. It will be recognized that the present non-invasive techniques are capable of detecting any reaction, disorder or disease so long as the biological material is present in the eye, more particularly below the conjunctiva of the eye. For example, and not by way of limitation, the inventors have discovered that cytokine-encoding polynucleotides show differing patterns in controls, presumably reflecting subclinical responses to a variety of adventitious contaminants. The pattern of cytokine expression in dry eye suggests a reduced overall immune response by comparison with controls but with an increased production of the neutrophil attractant chemokines CXCL-1, CXCL-2, CXCL-3, CXCL-5 and CXCL-6 but a reduced expression of another neutrophil attractant chemokine, IL-8. The result indicates a disease specific response that might be used as the basis of a diagnostic approach. Thus, elevated levels of . . . polynucleotide can be used diagnostically to detect a disorder or disease of the eye. By using the methods of the invention, it also is possible to quantify the severity of a reaction by measuring the levels of polynucleotides encoding cytokines when compared to a normal-standard sample.

The method for detecting a cytokine for distinguishing conjunctivitis from other reactions may alternatively employ the detection of other polynucleotides. For instance, the method might be used to detect changes relevant to a malignant process such as mutations in BRAF in cells, identification of bacterial 16S RNA, or identification of viral or protozoal products obtained from a subject suspected of having a melanoma. A sample from one patient with a conjunctival nevus demonstrated a markedly elevated proportion of RNA that did not code for protein. Much of this was micro RNA, important in the control of gene expression. These examples illustrate the wide utility of the approach. The levels of such cytokines are indicative of a reaction when compared to a normal standard cytokine polynucleotide profile in a similar tissue. Thus, the expression pattern of a polynucleotide will vary depending upon the type and degree of a reaction or disease. In this regard, the sample obtained, as described herein, may be used as a source to isolate polynucleotides.

Following eye sampling as described above, cells or cell fragments isolated from the conjunctiva may be lysed by any number of means, and other molecules may be obtained from the cells. These molecules, such as polypeptides or bacterial cell wall components, may then be detected and quantified using methods known to those of skilled in the art, for example by ELISA. Antibodies, in some cases monoclonal and in others polyclonal, to particular polypeptides or other molecules, can be used in immunoassays, such as in liquid phase or bound to a solid phase carrier, to detect polypeptides associated with a disorder, such as dry eye. In addition, the monoclonal antibodies used in these immunoassays can be detectably labeled in various ways. Examples of the types of immunoassays which can utilize monoclonal antibodies of the invention are competitive and non-competitive immunoassays in either a direct or indirect format. Examples of such immunoassays are the radioimmunoassay (RIA) and the sandwich (immunometric) assay. Detection of the polypeptide antigens using the monoclonal antibodies of the invention can be done utilizing immunoassays which are run in either the forward, reverse, or simultaneous modes, including immunohistochemical assays on physiological samples. Those of skill in the art will know, or can readily discern, other immunoassay formats without undue experimentation. In addition, there are a number of commercially available antibodies to cytokines of interest.

The term “immunometric assay” or “sandwich immunoassay” includes simultaneous sandwich, forward sandwich, and reverse sandwich immunoassays. These terms are well understood by those skilled in the art. Those of skill will also appreciate that antibodies according to the resent invention will be useful in other variations and forms of assays which are presently known or which may be developed in the future. These are intended to be included within the scope of the present invention.

Monoclonal antibodies can be bound to many different carriers and used to detect the presence of a cytokine polypeptide. Examples of well-known carriers include glass, polystyrene, polypropylene, dextran, nylon, amylose, natural and modified celluloses, polyacrylamides, agaroses and magnetite. The nature of the carrier can be either soluble or insoluble for purposes of the invention. Those skilled in the art will know of other suitable carriers for binding monoclonal antibodies, or will be able to ascertain such using routine experimentation.

In performing the assays, it may be desirable to include certain “blockers” in the incubation medium (usually added with the labeled soluble antibody). The “blockers” are added to assure that non-specific proteins, proteases, or anti-heterophilic immunoglobulins to anti-cytokine immunoglobulins present in the experimental sample do not cross-link or destroy the antibodies on the solid phase support, or the radiolabeled indicator antibody, to yield false positive or false negative results. The selection of “blockers” therefore may add substantially to the specificity of the assays.

It has been found that a number of nonrelevant (i.e., nonspecific) antibodies of the same class or subclass (isotype) as those used in the assays (e.g., IgG1, IgG2a, IgM, etc.) can be used as “blockers”. The concentration of the “blockers” (normally 1-100 μg/μl) may be important, in order to maintain the proper sensitivity yet inhibit any unwanted interference by mutually occurring cross reactive proteins in the specimen.

In another embodiment, the invention provides a kit for non-invasively obtaining samples from the eye comprising a cell collection device selected from the group consisting of a rigid surface, an adhesive tape, a porous structure or any combination of these and a cell lysis buffer suitable for preserving nucleic acids and/or other molecules in the eye sample. In another embodiment, the invention provides a kit comprising a collection device, a cell lysis buffer and a mRNA detection reagent for distinguishing allergic, irritant or inflammatory reactions and/or neoplastic abnormalities in a tissue. The kit comprises a polynucleotide detection reagent, for example, an oligonucleotide primer that is complementary to a polynucleotide sequence encoding a cytokine, such as IL-4 or a chemokine, such as CCL4. Such a kit may also include a preservative excipient and may be compartmentalized to receive in close confinement one or more independent containers such as vials, tubes and the like, each of the containers comprising one of the separate elements to be used in the method. If present, a second container may comprise a lysis buffer. The kit may alternatively include a computer chip on which the lysis of the cell(s) will be achieved by means of an electric current.

The kit may also have containers containing nucleotides for amplification of or hybridization to the target nucleic acid sequence which may or may not be labeled, or a container comprising a reporter, such as a biotin-binding protein, such as avidin or streptavidin, bound to a reporter molecule, such as an enzymatic, fluorescent, or radionuclide label. The term “detectably labeled deoxyribonucleotide” refers to a means for identifying deoxyribonucleotide. For example, the detectable label may be a radiolabeled nucleotide or a small molecule covalently bound to the nucleotide where the small molecule is recognized by a well-characterized large molecule. Examples of these small molecules are biotin, which is bound to avidin, and thyroxin, which is bound by anti-thyroxin antibody. Other methods of labeling are known to those of ordinary skill in the art, including enzymatic, fluorescent compounds, chemiluminescent compounds, phosphorescent compounds, and bioluminescent compounds.

Cells from the eye can be recovered non-invasively by using Duct tape (333 Duct tape, Nashua tape products), Scotch R tape (3M Scotch R 810, St. Paul, Minn.), or D-SQUAME (CuDerm, Dallas, Tex.). The conjunctiva or another part of the eye is stripped with the tape up to 5 times with one and the same tape. This tape stripping can be repeated up to 5 times with 5 different tapes. Additionally, it will be recognized that the stickier the tape, the fewer tape stripping(s) are required. The cells from the eye are recovered by vortexing and then centrifuging the tape(s) in an RNAse-free Eppendorf tube containing lysis buffer. The same lysis buffer can be reused for each piece of tape used at a single site on the eye. The entire procedure can be performed in less than 60 minutes.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

1. A biological material gathering device, comprising:
a microfiber;
an adhesive bead; and
a plurality of adhesive fibers;
wherein said adhesive bead is affixed to a first end of said microfiber; and
wherein said adhesive bead is covered by a plurality of adhesive fibers.

2. The invention of claim 1, further comprising:
a tonometer tip base; and
a combination housing;
wherein said tonometer tip base is affixed to a plurality of second ends of a plurality of said microfibers; and
wherein said combination housing surrounds said plurality of microfibers.

3. The invention of claim 2, further comprising:
an optical fiber;
wherein a first end of said optical fiber is affixed to said tonometer tip base.

4. The invention of claim 3, further comprising:
a fiber optic bundle;
wherein a first end of said fiber optic bundle is affixed to a second end of said optical fiber.

5. The invention of claim 4, further comprising:
a video imaging device;
wherein said video imaging device is affixed to a second end of said fiber optic bundle.

6. A biological material gathering device, comprising:
a microfiber;
an adhesive bead; and
a plurality of adhesive fibers;
wherein said adhesive bead is removably attached to a first end of said microfiber; and
wherein said adhesive bead is covered by a plurality of adhesive fibers.

7. The invention of claim 6, further comprising:
a tonometer tip base; and
a combination housing;
wherein said tonometer tip base is affixed to a plurality of second ends of a plurality of said microfibers; and
wherein said combination housing surrounds said plurality of microfibers.

8. The invention of claim 7, further comprising:
an optical fiber;
wherein a first end of said optical fiber is affixed to said tonometer tip base.

9. The invention of claim 8, further comprising:
a fiber optic bundle;
wherein a first end of said fiber optic bundle is affixed to a second end of said optical fiber.

10. The invention of claim 9, further comprising:
a video imaging device;
wherein said video imaging device is affixed to a second end of said fiber optic bundle.
